(19) United States
(12) Patent Application Publication
(10) Pub. No.: US 2005/0065592 A1
(43) Pub. Date: Mar. 24, 2005

(54) System and method of aneurism monitoring and treatment

(76) Inventor: Asher Holzer, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/667,328

(22) Filed: Sep. 23, 2003

(51) Int. Cl. A61F 2/06

(52) U.S. Cl. 623/1.13; 623/1.2; 600/486; 623/1.36

(57) ABSTRACT

An endoluminal stent device, system, and method enabling localized detection and treatment of adverse stent-related conditions. Embodiments of the present invention may enable monitoring of endoluminal pressure in the region of an aneurism, and/or compliance in the neck region of a stent device. Other embodiments of the present invention may provide systems and methods for treating a variety of aneurism conditions, by externally activating at least one expansion mechanism associated with the stent device at a desired future time after an initial stent placement procedure.

### FIELD OF THE INVENTION

The present invention relates to methods and devices useful in stent applications. Specifically, embodiments of the present invention relate to systems and methods of endoluminal stent monitoring and treatment.

### BACKGROUND OF THE INVENTION

The treatment of aortic aneurisms with endovascular stents or stent-graft prostheses is receiving increasing attention as an alternative to major abdominal surgery. Endoluminal stents may be inserted into a body using minimally invasive surgery. Such stents may be deployed by, for example, inflation of balloons or by self-expandable mechanisms such as Nitinol alloys, or by surgical attachment etc. The numerous advantages of stent graft treatments over open surgical repair of aneurisms include reduced blood loss, fewer days in intensive care, shorter hospital stays, fewer systemic complications, and shorter recovery time. However, there are various problems associated with stent graft treatments, for example, migration of stents, holes in the stent fabric, sudden ruptures, and endoleaks, all of which may occur unpredictably some time after the deployment of the stent.

Owing to the above-mentioned risks, patients with stent grafts are typically required to have regular checkups using, for example, CAT scans and angiograms. These check-ups are typically expensive and time consuming and may only randomly detect problems, e.g., by discovering symptoms related to stent cracking, aneurism monitoring, stent migration, and endoleaks, which cannot be anticipated by existing systems and methods. If check-ups are not performed frequently, potentially fatal developments may be overlooked.

### SUMMARY OF THE INVENTION

Embodiments of an aspect of the invention provide a stent device, for example, an endoluminal stent device, that enables localized detection and/or monitoring of adverse stent-related conditions such as stent cracking, stent migration, endoleaks, luminal pressure changes, stent movement, etc., for example, by monitoring pressure and/or compliance in regions of the stent device.

Some embodiments of the present invention enable aneurism monitoring by recording endoluminal compliance and optionally pressure measurements. Various sensors may be used, e.g., in a neck region of the stent, to determine, for example, compliance between the stent graft and a lumen wall, strain on the stent graft, expansion of the aneurism, pressure of the aortic walls on the stent graft, and the pressure the stent exerts on the aortic walls.

Some embodiments of the present invention enable prevention of aneurism expansion. Upon insertion of a stent graft, an expansion mechanism may be activated to expand the body of the stent to conform to the morphology of the aneurism. The expansion mechanism, which may include mesh, hooks, glue and polymers etc., may be bonded or anchored to the vessel wall in the area of the neck and/or body of the lumen hosting the aneurism, thereby preventing undesired wall expansion, leaking between the stent graft and the vessel wall, aneurism rupture, etc.

Embodiments of another aspect of the present invention provide various devices, systems and methods to control features of an implanted stent graft, for example, to correct adverse stent-related conditions, from a location external to a patient. An external stent activator may be used to induce activation of a mechanism associated with the stent device, for example, an expansion mechanism of the stent device may be triggered by energy generated from the stent activator. This may enable administering treatment remotely and non-invasively at a desired future time after the initial implantation of a stent graft, e.g., to strengthen the grip between the stent graft and a lumen wall, to increase pressure between the stent graft and the vessel wall, or to stop blood leaks along the stent etc.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic block diagram of a stent system, according to some exemplary embodiments of the present invention;

FIG. 2 is a schematic illustration of a stent device including a body expansion mechanism deployed in a lumen in a non-expanded position, according to some exemplary embodiments of the present invention;

FIG. 3 is a schematic illustration of the stent of FIG. 2 with the body expansion mechanism in an expanded position, according to some exemplary embodiments of the present invention;

FIG. 4 is a schematic illustration of a stent device including an anchoring mechanism integrated into a body expansion mechanism, according to an exemplary embodiment of the present invention;

FIG. 5 is a schematic illustration of a stent device including a neck expansion mechanism in an expanded position, according to an exemplary embodiment of the present invention;

FIG. 6 is a schematic flow chart illustrating a method for monitoring endoluminal pressure and compliance, according to an exemplary embodiment of the present invention.

FIG. 7 is a schematic illustration of a sensor for use in a stent device according to an exemplary embodiment of the present invention;

FIG. 8 is a pictorial illustration of a micro machined pressure sensor for use in a stent device according to some embodiments of the present invention;

FIG. 9 is a schematic perspective view of a J-shaped pressure sensing apparatus mounted on a stent device according to some embodiments of the present invention;

FIG. 10 is a cross-sectional schematic illustration of a stent device including a strain measurement mechanism according to an exemplary embodiment of the present invention;

FIG. 11 is a top view schematic illustration of a stent device including a coiled tube mechanism, according to an exemplary embodiment of the present invention; and

FIG. 12 is a schematic illustration of a spring mechanism for use with a stent device, according to an exemplary embodiment of the present invention;

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The word “endoluminal” as used herein may refer to internal lumen, orifices, vessels, channels, cavities, organs, aneurisms, etc., and may encompass all endoluminal and endovascular regions. The term “vessel” or “host vessel” may refer to any endoluminal vessel wherein a stent graft may be placed, for example, the aorta. The expression “stent graft” as used herein may refer to any device used to support a bodily orifice, vessel, or cavity etc. The stent graft may be a thread, rod, or catheter, etc., inserted into a tubular structure or lumen, for example, a blood vessel.

Embodiments of an aspect of the present invention enable localized detection of adverse stent-related conditions by monitoring, for example, endoluminal pressure, strain and compliance, to provide indications of various effects, for example, stent weakening, stent migration, endoleaking (e.g. the leakage of blood into the aneurism), rupture status, aneurism growth, etc. Embodiments of another aspect of the present invention enable external operation or manipulation of a stent, for example, to treat adverse stent-related conditions, at a desired future time after initial stent graft implantation. Adverse conditions may be indicated, for example, by changes in endoluminal pressure, changes in the aneurism volume, and/or changes in compliance between the stent and the blood vessel.

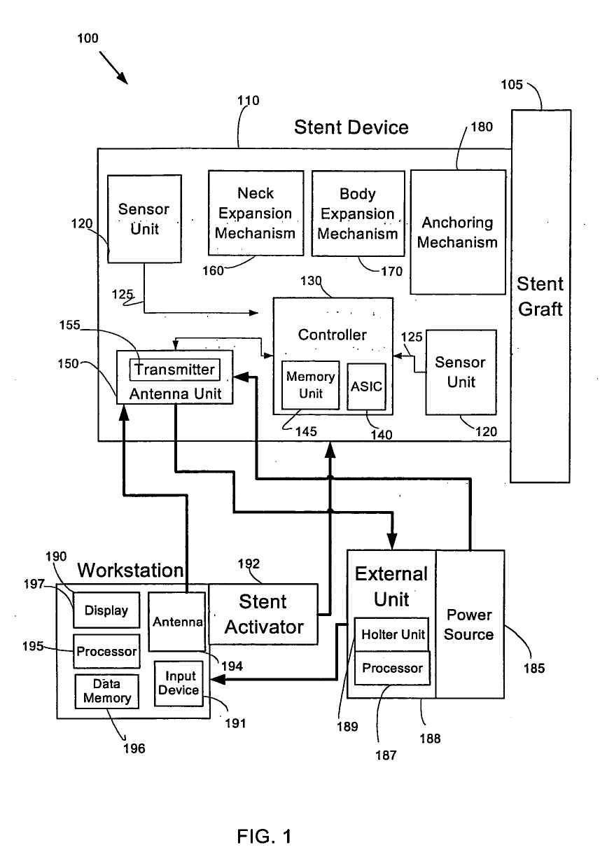

Reference is now made to FIG. 1, which is a schematic illustration of a stent system 100 according to some exemplary embodiments of the present invention. As can be seen in FIG. 1, stent system 100 includes a stent device 110, which may include a stent graft 105 for implantation into a vessel or lumen, e.g., to create an endovascular pressure seal that prevents flow around the stent graft into the aneurism. Stent graft 105 may enable isolating the aneurism from the blood circulation. According to exemplary embodiments of the invention, Stent device 110 may enable endovascular monitoring and/or treatment of an aneurism, as described below.

System 100 may include an external data reception and/or processing unit 188, to receive, process and/or display transmitted aneurism or stent graft data from stent device 110. In some embodiments of the invention, system 100 may include an external stent activator 192, to enable external activation of mechanisms in stent device 110. In some embodiments of the invention, system 100 may include a workstation 190, which may include a computing system. Workstation 190, stent activator 192 and external unit 188 may be implemented as independent units, or may be combined in a single external unit or any combination of units incorporating any suitable combination of hardware and/or software to perform the functions described herein. Stent activator 192 may be activated and/or controlled by workstation 190, for example, to externally or remotely operate stent device 110, for example, by transmitting energy and/or signals to device 110.

Stent Device 110 may include one or more sensing units 120 to measure endovascular pressure, strain, compliance, or other relevant parameters, as described below. Sensors 120 may provide signals responsive to predetermined parameters to a controller 130 via, for example, a conductor 125, for example, to identify and/or quantify pressure at a predetermined vicinity of device 110 and/or compliance between device 110 and a lumen wall, for example, at an aneurism of a blood vessel. Controller 130 may include a microprocessor or CPU, a memory unit 145 and optionally an ASIC 140. Controller 130 may instruct an antenna unit 150 to transmit data, including data based on the signals from sensor units 120, to external reception unit 188. For example, antenna unit 150 may send alert signals via transmitter 155 to reception unit 188, when the parameters sensed by sensor units 120 meet predetermined criteria, as described below. Stent device 110 may include one or more neck expansion mechanisms 160, body expansion mechanisms 170, and/or anchoring mechanisms 180. Stent device 110 may be connected to or integrated with stent graft 105.

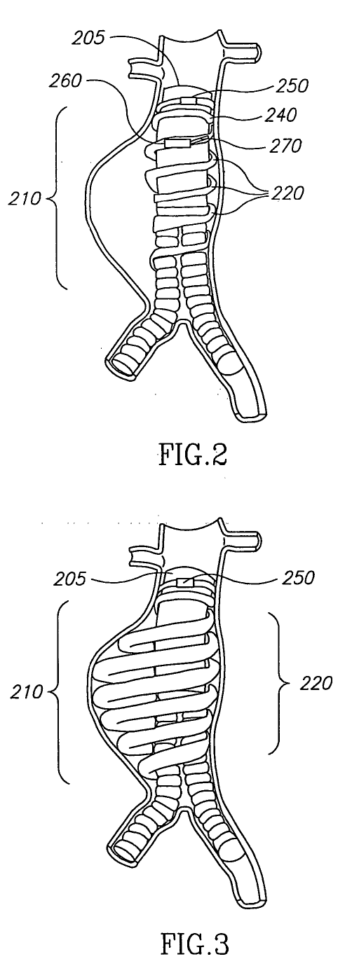

Reference is now made to FIG. 2, which is a schematic representation of a stent device 210 according to an exemplary embodiment of the invention. Stent device 210 may include a stent graft 205, and may further include a body expansion mechanism 220 and/or a neck expansion mechanism 240. Expansion mechanisms may include, for example, expandable tubes or coils and/or expandable rings or bands. For example, mechanisms 220 and/or 240 may include an expandable tube shaped element that may be wrapped around stent graft 200 like a coil, or otherwise associated with stent graft 200. FIG. 2 shows mechanisms 220 and 240 in a non-expanded position. Stent device 210 may include one or more sensor units 250, which may provide signals responsive to measured parameters to controller 260. Stent device 210 may include an antenna 270 to transmit signals to an external unit such as reception unit 188 (FIG. 1). Controller 260 and antenna 270 may be located at any location within stent device 205.

Reference is now made to FIG. 3, which is a schematic representation of body expansion mechanism 220 in expanded position, according to an exemplary embodiment of the invention. Body expansion mechanism 220 may enable stent device 210 to conform to the shape and to the morphology of a host vessel (e.g., the vessel that hosts the stent), such as the aorta, at one or more locations in the neck and/or body of the aneurism. Body expansion mechanism 220 may be activated at the time of stent insertion or at a later time, for example, in response to an externally activated trigger, to conform to the morphology of an endovascular area, as shown schematically in FIG. 3. Mechanism 220 may be sufficiently flexible to enable the size and shape of stent device 210 to be adapted to the particular in-vivo morphology hosting stent graft 205, and may not be limited to a predetermined uniform expansion size. Other suitable mechanisms may be used.

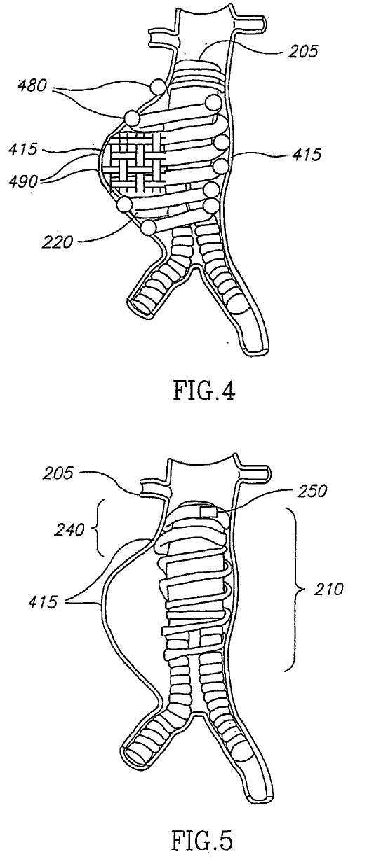

Reference is now made to FIG. 4, which is a schematic representation of the stent device of FIG. 1 with anchoring mechanism 180 (FIG. 1) in an activated position, according to an exemplary embodiment of the invention. Anchoring mechanism 180 may be utilized to anchor stent graft 205 to a lumen wall 415, for example, in the area of the neck and/or body of the lumen hosting the aneurism. Anchoring mechanism 180 may include, for example, one or more hooks 480, and a mesh 490, for example, a mesh formed of a suitable metal, which may be urged against and/or at least partly embedded in the aneurism wall. Metal mesh 490, for example, may extend from a portion of body expansion mechanism 170 (of FIG. 1), and may be pressed against the lumen wall 415, thereby providing a support to lumen wall 415. Anchoring mechanism 180 may be anchored into or onto lumen wall 415 using glues, polymers etc.

Anchoring mechanism 180 may be used to fill the space between the lumen wall 415 and stent graft 205, thereby attaching stent graft 205 to the lumen wall 415, optionally at various points along the cross-sectional circumference of the lumen wall, along the entire cross-section of the lumen wall, or along a portion of the lumen wall cross-section. Anchoring mechanism 180 may be activated at the time of insertion of stent device 110 (of FIG. 1) into the lumen or vessel, and/or at a later time, for example, following insertion of stent device 110, using an external energy or signal transmission. The anchoring mechanism may prevent, for example, stent migration, by holding or fastening the stent to the lumen wall 415. Further, anchoring mechanism 180 may prevent growth of aneurism tissue, by connecting the aneurism to the stent graft. Further, anchoring stent device 110 to lumen wall 415 may aid rupture prevention of aneurism due to blood flow between stent graft and lumen wall.

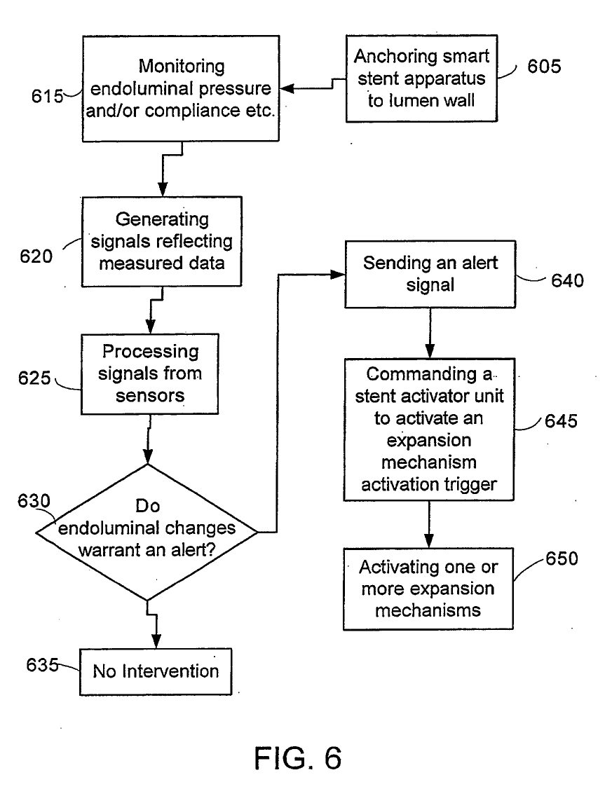

Reference is now made to FIG. 5, which is a schematic representation of the stent device of FIG. 1 with an expanded neck expansion mechanism 240 in stent device 210, according to an exemplary embodiment of the invention. Neck expansion mechanism 240 may be integrated into stent device 210, for example, to enable the neck of stent graft 205 to form a pressure bond or a pressure seal with the host vessel wall 515, such as the aorta. Such a pressure seal formed with the vessel wall may prevent blood flow around the stent graft 205 into an aneurism. The neck expansion mechanism 240 of stent device 210, or a part of device 210, may be activated at the time of insertion of the stent, and/or at a later time by using an external energy transmission or signal etc. Such a mechanism may enable the size of the stent graft neck to be adapted to the particular in-vivo conditions, for example, at a designated future time after stent insertion, e.g., when a leak has been diagnosed, for example, by a CT scan, leak sensor, or compliance sensor etc. Other suitable mechanisms may be used. Neck expansion mechanism 520 may include expandable rings or coils that may be integrated into the upper distal and lower proximal neck regions of stent device 510. For example, proximal and distal inflatable cuffs may be provided.

According to an aspect of embodiments of the present invention, as illustrated in FIGS. 1-5, sensor units 120 (FIG. 1) may be placed at one or more necks (e.g., distal, proximal and/or other ends) of stent graft 105 (FIG. 1), and/or in one or more locations on the body of stent graft 105, between the ends of stent graft 105. One or more of sensor units 120 may include one or more pressure sensors, compliance sensors, strain sensors, or any other suitable sensors.

According to an aspect of an embodiment of the present invention, controller 130 of FIG. 1 may enable control of stent device 110. Although the invention is in no way limited in this regard, controller 130 may include an Application Specific Integrated Circuit (ASIC) 140, to enable implementation of application specific functions. ASIC 140 may provide a single-chip solution that may be relatively space-effective. The electronics in controller 130 may include circuits that amplify and transform raw data from sensors 120 into a robust signal (e.g., a front-end circuit), circuits that handle the transmission of the data from the stent to the outside world (e.g., a transmission circuit). The necessary electronics for the front-end may be integrated into a silicon area, for example, of less than 0.5 mm². The size of the transmission circuit may depend on external components like capacitors. The type or capacitance of capacitors in the circuits may depend on the energy supply, such as batteries, RF, ultrasound generation, etc., as well as the carrier frequency used for communication. An example of an ASIC that may be integrated into stent device 110 is the SensoNor ASIC, which is commonly used with the tire pressure sensor SP30, from SensoNor (Horten, Norway). This device is based on the SP12 device, and may add a level of integration in order to meet market demand for flexible, customer specific behavior, and solutions. Such an ASIC, for example, may have a micro-controller that may be programmed for custom-made measurements and read-outs, and LF-input stages. An evaluation kit may be included. ASIC 140 may implement radio-frequency read-out. Any other suitable implementation of controller 130 using any desirable combination of hardware and/or software may be used.

Controller 130 may be associated with a memory unit 145 wherein data may be stored. Data may include, for example, patient data, stent data, aneurism data, sensory data, executable code, and other relevant data. The controller may include a telemetry circuit, to measure, transmit, and receive data from/to components of stent device 110, and to receive remote activation signals from external workstation 190, for example to trigger one or more of expansion mechanisms 160 and/or 170. CPU 130 may process signals representing sensed data received from sensor units 120. CPU 130 may determine whether signals representing sensed data are indicative of relevant or substantial endoluminal changes, such as, for example, increased pressure, decreased compliance, or other relevant parameters. In an alternative embodiment, an external controller 187, e.g., a processor, for example, in reception unit 188 may process the signals representing sensed data, and determine when received signals may require providing a user of system 100 with an alert.

In the case where controller 130 determines that an endoluminal change warrants sending an alert to a user, controller 130 may instruct a transmitter 155 to transmit a signal, for example, an alert signal, to a reception unit 188. Antenna unit 150 may be used to transmit the signals. For example, controller 130 may receive a signal representing a value of sensed pressure from one or more of sensor units 120. The measurements may be scaled, for example, to an initial pressure reading. For example, it may be expected that the blood pressure in the aneurism initially has a certain value, and that in the vessel (e.g., aorta) the blood pressure has a different value. If the pressure in the aneurism either absolutely or relative to the aortic blood pressure changes substantially from its initial value, e.g., by a predetermined threshold, a leak may be suspected and the alarm or alert signal may be activated.

According to an aspect of an embodiment of the present invention, transmitter 155 and/or antenna unit 150, which may include any type of in-vivo antenna known in the art, may send a signal, such as a wireless signal, to reception unit 180. Antenna unit 150 may also be configured to receive signals from outside a patient's body. For example, antenna unit 150 may receive wireless signals incorporating commands originating from reception unit 188, stent activator 192 and/or workstation 190. According to an embodiment of the present invention, antenna unit may be adapted to receive energy from an external power source 185. For example, power source 185 may include an electricity source, e.g., a battery, a solar cell, or any other power source. Power source 185 may transmit energy, for example, by wirelessly transmitting electromagnetic energy, e.g. radio frequency (RF) energy, to antenna 150. The energy may be induced via antenna unit 150, and may power stent device 110.

Additionally or alternatively, an independent power source, for example a battery, may be included in stent device 110, providing energy to the implanted sensors and sensor related circuitry. Another embodiment may combine, for example, an internal energy source with less power than required to be operated alone, with some of the energy being provided by transmission from an external energy source. Additional or alternative energy sources may include, for example, remote powering by radio frequency (RF) radiation, remote powering by ultrasound, remote powering by two inductively coupled coils, micro-batteries, micro combustion using liquid fuel, with storage capacity of hundreds of times more energy than state-of-the-art batteries, micro fuel cells, and micro power stations with turbines utilizing the blood flow supplied by the beating heart.

According to an aspect of an embodiment of the present invention, the stent graft structure or a part thereof, which may include metal components and/or a wire attached to the stent structure, for example, as used in the stent device 110, may be used as an antenna. For example, an 8 cm long stent, and the possibility of wrapping an expansion mechanism with coiled around it, may effectively enable usage of very low frequency waves for delivering energy, as well as for telemetry, if desired.

According to an aspect of an embodiment of the present invention, reception unit 188 may include a monitoring apparatus, such as a Holter unit or apparatus 189, which may enable, via telemetry for example, continual or periodic in-vivo monitoring. Reception unit 188 may be external to a user's body, and may be worn or carried by a stent user or may be positioned in the vicinity of a stent user. For example, reception unit 180 may be located on or in a mobile device, such as a watch, a mobile phone, palm pilot like device, and may be borne or suspended on clothing, may be located in a car, on a bed, or in any location where it may receive alert signals from antenna unit 150.

Reception unit 188 may be powered by power source 185, which may include an internal and/or an external power source unit to provide power to reception unit 188 and/or stent device 110 as described above. Reception unit 188 may include a processor 187 to process received signals and determine whether received signals warrant sensing of an alert for a user.

Stent activator unit 192 may activate anchoring mechanism 180, neck expansion mechanism 160, and/or body expansion mechanism 170. A medical professional, for example, may initiate remote or external operation or manipulation mechanisms of stent device 110. The medical professional may initiate external operation a predetermined period after installation of stent device 110, and/or based on monitoring of data received from sensor units 120 and/or in response to an alert signal received from device 110.

To activate one or more of mechanisms 160, 170 and 180, for example, stent activator unit 192 may transmit energy to stent device, for example, using heat, acoustic signals, data signals etc., which may trigger the activation of one or more of mechanism 160, 170 and/or 180. These mechanisms may be activated independently, in unison, or in any combination. Other suitable triggering mechanisms may be used by stent activator unit 192, as described below. Stent activator unit 192 may be independent of workstation 190, or may be operationally connected to workstation 190. Stent activator 192 may enable activation of mechanisms 160, 170 and/or 180 upon installation of stent device 110, or at a desired time after installation of stent device 110.

For example, expansion mechanisms may be initiated, for example, by boiling or vaporizing a liquid having a predetermined boiling point, for example, around 46 degrees centigrade, thus expanding a non-collapsible scaffold, as is known in the art. Expansion mechanisms may be expanded, for example, by providing heat to, for example, a shape memory alloy (e.g., Nitinol-based structure), which, upon exposure to the heat, may expand to a pre-configured shape. Expansion may alternatively be activated, for example, by breaking a disc between two chambers in a tube or ring etc., using, for example, heat energy, ultrasonic energy etc. The disc may, for example, separate between different materials, which when mixed, may initiate a chemical reaction that activates expansion of a tube or ring etc. Other suitable mechanisms may be used, including radiators, magnetic activators, ultra-sonic activation, RF decoding signals, or other suitable mechanisms. In other embodiments, stent activator unit 192 may generate at least one signal to activate neck expansion mechanism 160. The signal may be transmitted to stent device 110, and may be received by antenna 150. Antenna 150 may send the received signal to CPU 130. CPU 130 may send a signal to neck expansion mechanism 160 to activate the expansion mechanism, thereby closing a gap or creating a pressure seal between the neck of stent device 110 and the lumen, etc.

Workstation 190 may externally activate one or more expansion mechanisms in stent device 110. Workstation 190 may include an antenna unit 194, or a transmitter unit, to transmit signals to stent device 110. Antenna unit 194 may include a radiator unit, which may be used to transmit energy to stent device 110, for example, via antenna unit 150. Antenna unit 194 may enable charging of a power storage unit located in stent device 110. Such a radiator unit may be located within antenna unit 194 or external to antenna unit 194. For example, such a radiator unit may be located under the patient, to enable effective energy transmission to stent device 110 within the patient.

Workstation 190 may include one or more input devices 191, a processor 195, memory unit 196, display apparatus 197, and any other components that may aid the external operation of stent device 110. Workstation 190 may be separated into several independent sub-systems optionally placed in deferent locations.

In the case where reception unit 188 receives an alert from antenna unit 150, a user may verify whether the alert requires further attention, for example, by performing a CAT scan or angiogram etc. In the cases where it is determined that the aneurism requires stent graft adjustment, remote adjustment may be initiated by stent activator unit 192. In the cases of, for example, stent migration or endoleaking, etc., a medical professional, for example, may determine that a neck expansion of stent device 110 is required. In such a case, the medical professional may instruct the stent activator 192 to activate neck expansion mechanism 160.

In the cases of, for example, stent weakening, migration, rupturing, or endoleaking etc., a medical professional, for example, may determine that activation of body expansion mechanism 170 may be required. The medical professional may use an input device 191 to instruct stent activator 192 or another relevant controlling mechanism, such as processor 195, optionally located in workstation 190, to generate a trigger to activate one or more expansion mechanisms to better seal the stent to a lumen wall, for example, using one or more trigger mechanisms described above. Other suitable input mechanisms may be used.

To produce stent device 110, suitable mechanisms and/or circuits, as described in detail above and below, may be integrated with stent graft 105, for example, such that at least one expandable mechanism is integrated into stent device 110. For example, an expandable tube or ring may be wrapped around the stent body and/or neck. Reference is now made to FIG. 6, which is a schematic flow chart illustrating blocks representing various operations that may be performed by the stent system 100 of FIG. 1, according to an aspect of some embodiments of the present invention. At block 605, upon insertion of stent device 110 into a bodily vessel, the stent device may be anchored to a lumen wall, by activating one or more of the expansion mechanisms as described above. For example, anchoring mechanism 180 may be activated to anchor stent device 110 to the lumen wall. Body expansion mechanism 170 may be activated, with or without anchoring mechanism 180. At block 615, endoluminal pressure and/or compliance may be monitored, for example, continuously or periodically, using one or more of sensor units 120. At block 620, sensor units 120 may generate signals reflecting measured data and may send these signals to controller 130.

At block 625 controller 130 may process signals from sensors 120, to determine if there are endoluminal pressure and/or compliance changes. At block 630, controller 130 may determine whether endoluminal pressure changes, compliance changes, and/or alternative endoluminal changes, warrant an alert. Such a decision may be determined according to pre-determined criteria, which may be stored in memory unit 145. Determination of alerting conditions may be made by a controller located in reception unit 188. In the case where no alert is warranted, at block 635 no alert is sent from stent device 110, and therefore no intervention is required. In the case where an alert is warranted, at block 640, controller 130 may instruct transmitter 155 to send an alert signal via antenna unit 150 to reception unit 188.

At block 645 a medical practitioner may operate stent activator 192 located outside of a body to activate at least one of the expansion mechanisms, such as neck expansion mechanism 160, body expansion mechanism 170, and/or anchoring mechanism 180 in stent device 110. Activation of such a mechanism may require generation of an expansion mechanism activation trigger, for example, by using heat, acoustic signals, data signals etc. to externally activate at least one of the expansion mechanism. At block 650, by using such an activation trigger, stent device 110 may be externally operated, for example, by inflating an expansion mechanism, thereby providing treatment to an aneurism using stent device 110. Any combination of the above steps may be implemented. Further, other steps or series of steps may be used. According to an embodiment of the present invention, an external power source may transmit energy using Radio Frequency (RF) signals to antenna unit 150, thereby providing power to stent device 110.

According to an aspect of some embodiments of the present invention, one or more of sensor units 120 may include one or more compliance sensors, for example, to measure the tightness or fit between the stent device 110 and a lumen wall 415 (FIG. 4), for example, according to an indication of force or pressure. Such sensors may enable measurement of the force applied on stent device 110 and/or a lumen wall 415, the strain on stent device 110 and the lumen wall 415, and the relative pressure exerted on stent device 210 and/or the lumen wall 415. For example, sensor unit 120 may measure compliance by determining differential pressure between the aneurism and the inside of the graft, possibly at a plurality of locations. Absolute and differential pressure sensors, for example, may be attached to stent graft 105, or may be integrated with stent graft 105, for example, penetrating the graft structure. Since the composition of the material inside an aneurism may change over time, for example, due to blood clotting and development of the clots into body tissue, the material may be relatively inhomogeneous. The use of a plurality of sensors in different locations operating in parallel may enable greater measurement accuracy. Various methods known in the art of measuring compliance may be used, for example, using force or pressure to determine displacement.

For example, a certain change in the measurement of compliance may provide an indication of rupturing of an aneurism, aneurism growth, stent graft weakening, stent graft migration, etc. According to an embodiment of the present invention, one or more of sensor units 120 may be adapted to measure blood pressure pulsation comparison.

Sensor units 120 may, for example, measure strain in a ‘tube’ attached to stent graft 105 (inside or outside), strain on a metal structure, e.g., a spring, on stent device 110, and strain on the stent graft 110, etc. In some embodiments of the invention, sensor units 120 may measure additional parameters, for example, blood freshness according to oxygen content, electrochemical detection, Fe content, ion content (charged particle detection), and the intensity and shade etc. of the blood color. Electrical conductivity may be measured by sensors 120, as may, for example, moisture or fluidic content. The rate of blood flow in the aneurism may also be measured, for example, thermally, electro-magnetically, by using a narrow tube section, or by other suitable means, as are known in the art.

The placement of the sensing elements in stent graph 105 may require attachments so as not to ‘float’ freely in the aneurism. Such attachment may take place, for example, in the manufacturing process of the stent graft or during the insertion procedure. In the manufacturing process, for example, stitching, sewing or gluing may be used. In the case where placement of the sensor(s) inside the aneurism and/or on stent graft 105 may require rotational orientation of stent graft 105, the stent graft may be inserted into a saccular aneurism. In the case where sufficient rotational control of the stent graft cannot be assumed, stent device components may be attached inside the stent graft. In the case where the stent device components are attached inside the stent graft, a series of pressure sensors, for example, 4 pressure sensors separated by a rotational 90 degrees each, may be used. In order to minimize the volume of the stent system inside a stent graft sheath, sensors may be distributed along the sheath length direction so as not to overlap one another.

According to an aspect of some embodiments of the present invention, an absolute (e.g., direct) pressure measurement apparatus may be used in stent device 110. Such a pressure measurement apparatus may not require a connection (e.g., ‘pipeline’, ‘tube’, etc.) between the sensing element and a target site. In such a case, the connection between the stent graft 105 and the sensor 120 may be relatively stiff, thereby limiting the influence of the heart beat, body bending etc. Such an implementation of sensing apparatus may be inserted during a normal insertion procedure. In the case where differences exist between the outside pressure and inside pressure of stent graft 105, intelligent measurement protocols and/or signal conditioning may be used to provide adequate signals, as are known in the art. Measurements of the various sensors may be calibrated at the time of deployment in the body. Any changes relatively to the first measurement may indicate a possible leak, change in compliance, etc. Calibration at time ‘zero’, for example, may enable the measurement of relative values instead of or in addition to absolute values.

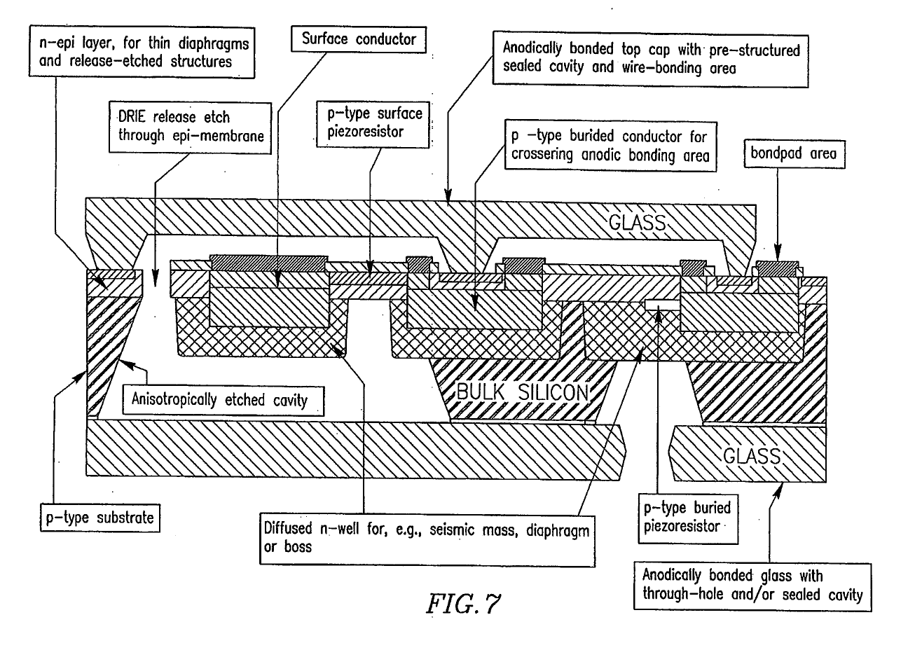

In the case where differential pressure is required as output, such an output may be achieved by using absolute sensors. However, since two sensors never have identical properties, with respect to sensitivity, offset, drift, etc., similarly behaving components may be paired up. More elaborate characterization procedures may be used, such as an automobile tire pressure sensor, for example, as provided by SensoNor. Such a sensor may enable usage of optimally matched components, thus reducing possible errors. An example of a sensor apparatus that may be used within stent device 110 can be seen with reference to FIG. 7, which schematically illustrates a cross-section of an exemplary sensor device, showing an accelerometer as well as an absolute pressure sensor. Other elements or combinations of elements may be used.

According to an aspect of some embodiments of the present invention, stent device 110 and/or components of stent device 110 may be miniaturized to provide considerable area reduction, and thereby enable use of selected components or functions in a plurality of vessel sizes, lumen sizes, stent devices etc. Miniaturization techniques may include use of single-chip technology, surface micro-machining, wafer thinning, and/or any other suitable techniques.

In particular, single-chip technology may be used when there is a need for an electronics chip or element inside a vessel. A single-chip solution may be provided with a sensor area and CMOS area on the same side of the same silicon chip, produced from the same wafer. Alternatively, a single-chip solution may be provided by bonding a CMOS chip and sensor chip from two different silicon wafers to each other (e.g., on the chip level or wafer level). The bonding could be, for example, by flip-chip or fusion, or by other suitable means.

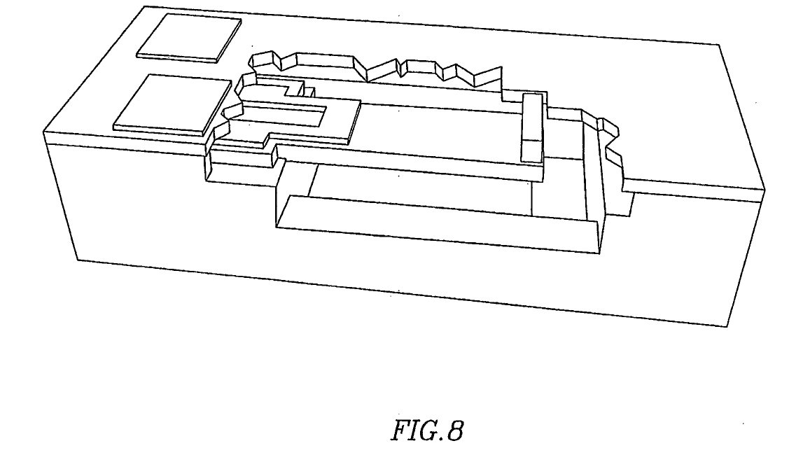

According to an aspect of an embodiment of the present invention, a miniature piezo-resistive surface micro-machined silicon pressure sensor may be used. An example of such a sensor is used in the PressureWire product by RADI Medical Systems AB (Uppsala, Sweden), for use in catheter based medical equipment for intravascular pressure measurements. Reference is now made to FIG. 8, which illustrates an example of RADI's pressure sensor. The RADI component as illustrated may be as narrow as 150 micrometers in the currently smallest in-silicon-plane dimension, and may include a pressure sensing membrane. Other dimensions and components may be used.

According to an aspect of some embodiments of the present invention, alternative small and low power sensors for medical applications may be used in apparatus 110, including, for example, the sensors described in, G. Bistué et al, “A micromachined pressure sensor for biomedical applications”, J. of Micromec. and Microeng., Vol 7, No 3, September 1997, pp 244-246; H.-L. Chau, K. D, “An Ultraminiature Solid-State Pressure Sensor for a Cardiovascular Catheter”, IEEE Trans. Elec. Dev., Vol 35, No 12, December 1988, pp 2355-2362; C. Hierold et al., “Low power integrated pressure sensor system for medical applications”, Sensors and Actuators 73 (1999), pp. 58-67; and in D. Goustouridis et al., “Ultraminiature silicon capcitive pressure-sensing elements obtained by silicon fission bonding”, Sensors and Actuators A 68 (1998), pp 269-274.

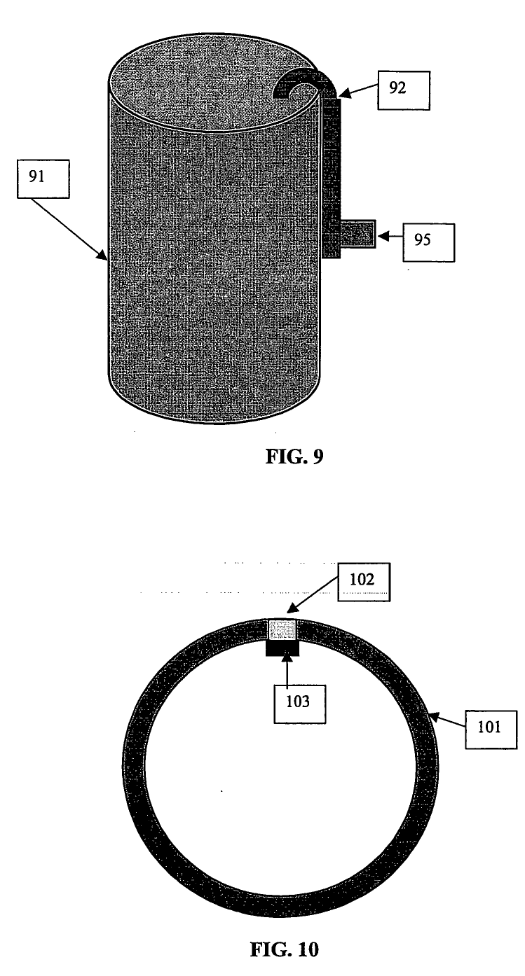

According to aspects of some embodiments of the present invention, differential pressure sensing may be implemented to enable differential pressure measurement. For example, by using two or more measuring or monitoring locations, each of these points having, for example, a membrane that responds to pressure changes and transfers the pressure change through a medium within the device or connection. Such membranes are known in the art. For example, one of the measuring locations may be in the aneurism. The connection to another site may be, for example, by means of a “J-shaped” (without the upper horizontal line on the J, and upside down) generally solid tube, that goes from the membrane in the aneurism alongside the stent graft through the neck area, and bends over the top of the stent graft into the blood flow inside the graft. The opening may face down, to help by providing a flushing action, and/or to make sure the measurement is not influenced by the vessel wall. As can be seen with reference to FIG. 9, a J-shaped connection solution may be provided, where a stent graft 91 is connected to a pressure-sensing element 95 by a connection element 92. The J-shaped connection solution avoids making holes through graft 91, and may also help avoid using graft 91 as a membrane. The J-shaped configuration, or other similar configurations, may enable measurement of relative pressure between a side of an aneurism and the intra aortic blood pressure. Other shaped pressure measurement configurations may be used.

The connection to the other measurement location may alternatively be by means of going directly through stent graft 91, either by a hole in the graft into which the connecting channel is placed, by the differential pressure sensor itself being terminated by a membrane on each side, and/or by using graft 91 itself as the outer sensing membrane on one side of sensor 95. The silicon-sensing device itself may be placed in the aneurism, where there may be suitable space. In some embodiments, the sensing device may be attached directly to the stent graft, e.g., on the outside of the graft or inside the graft hole.

According to an aspect of an embodiment of the present invention, a sensor connection may be embedded in the stent graft. In this way, the outer surface may be made as smooth as required for a tight fit, and the stent may remain as the outer layer. This may be important, for example, where the stent is to be attached to the vessel walls. The connection may be expanded during manufacturing, and/or after insertion, either partially and/or wholly. After insertion, the connection may be completely filled and sealed. Alternatively, a pre-filled connection may be used and may, for example, be flattened during insertion and then expanded after insertion. Alternatively, a sensing tube may be inflated after the deployment of the stent graft, while it is already in place. Such a solution may increase the tightness of the grip of the neck to the vessel, and provide the stent system with therapeutic capabilities in addition to monitoring capabilities.

According to an aspect of some embodiments of the present invention, as can be seen in FIG. 1, sensor unit 120 may, for example, measure compliance of stent graft 105 to a lumen, or strain on stent graft 105, etc. Sensor unit 120 may be placed inside stent graft 105, for example, by being inserted into a small pocket sewn onto the inside of the graft. Compliance may be measured by, for example, using a tube filled by a fluid or gas, on the outside of stent graft 105. In this way, the tube may be squeezed between the stent graft 105 and the vessel wall. When inserted, the cross-section of the tube may be squeezed to a roughly oval shape. If, over time, the vessel wall yields, this may result in a less oval, more round, cross-section, which in turn may reduce the pressure.

According to an aspect of some embodiments of the present invention, compliance may be measured by using a sensor connected to a pressure-sensing chamber located between two sections of a squeezable tube. Such a tube may be made from a relatively soft material that may be crimped along with the stent graft and inserted into a stent sheath. For example the tube may be plastic-based. The tube may be partially filled before it is released from the sheath, as the required volume may be considerable because the length of the tube may be on the order of the circumference of the vessel. According to an embodiment of the present invention, the tube may be filled by inserting a liquid with a sufficiently low boiling point before shutting the tube. When the tube is inserted into the human body, the increased temperature may make some of the liquid evaporate, thereby filling the tube with a gas of reasonably constant pressure. According to an embodiment of the present invention, salts may be inserted into the tube before shutting it, and thus the tube may be filled by osmosis. In such a case the tube may be made out of semi-permeable material, such as, for example, a thin silicon film.

According to an aspect of an embodiment of the present invention two thin tube parts with a hinge between them may be used. The two parts may be filled before delivery, be placed inside the sheath in their length direction, and placed after release.

As can be seen with reference to FIG. 10, an example is illustrated of a strain measurement mechanism using a tube, according to an aspect of an embodiment of the present invention. Stent graft 101 may be equipped with a container 102, in which pressure may be measured, using a tube-based pressure-sensing element 103. The apparatus illustrated in FIG. 10 may enable measurement of expansion, as a way of determining strain or compliance. According to an embodiment, a harder tube may be used, which may enable gating the measurements from the heart.

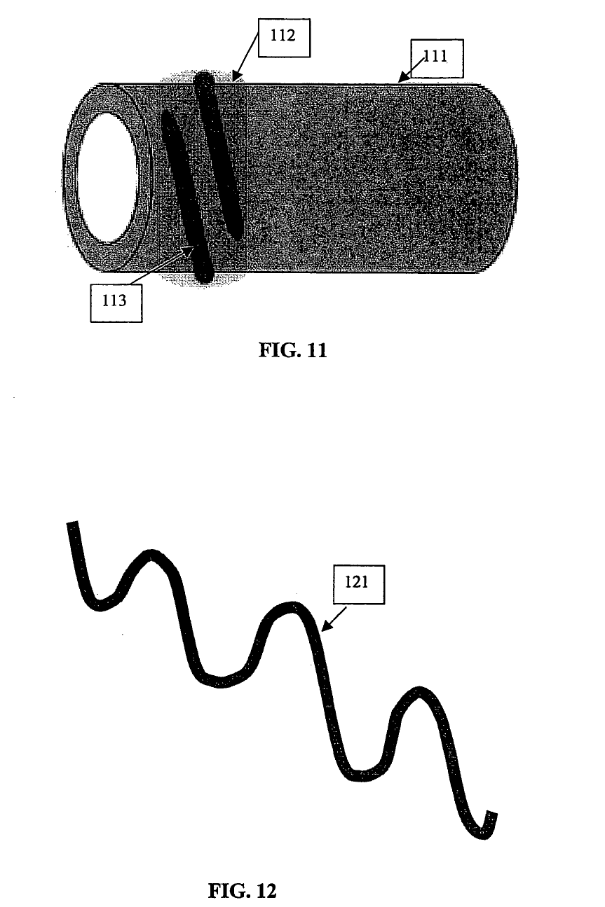

According to an aspect of an embodiment of the present invention the tube may be shaped as a coil, such that the tube rolls around the stent graft at various points. As can be seen with reference to FIG. 11, a coil 112 may be inserted into a bag 113 on the outside of a stent graft 111. The bag may have relatively stiff walls, to aid prevention of leaks.

According to another aspect of an embodiment of the present invention, sensor unit 120 may be placed in a tubular spring structure, coiled around stent graft 111. Such a structure may be expandable according to the size of the target vessel. Such a structure may be more easily filled before delivery. The bending parts of the spring structure may be formed of (e.g., hollow) metal, enabling a spring-like function. The spring structure may be skewed in order to diminish the overlap between sections inside the sheath, therefore taking up less space in the sheath. As can be seen with reference to FIG. 12, the spring structure 121 may ‘hang’ in a spiral around a stent graft.

According to another aspect of an embodiment of the present invention, sensor unit 120 may be placed inside a tube attached to the inside of the stent graft. Such a tube may go all the way around the entire circumference, or may be intersected by a container. Such a container may be filled by a substance, such as silicone oil or any other substance enabling pressure to be measured by a pressure sensor, which may be located, for example, on the inside of the graft or at other suitable locations. When the vessel or the aneurism expands, the tube may be lengthened, and the pressure in the container decreased.

According to the above-described embodiments, for applications requiring significant space, sensors may be inserted in the aneurysm area, where there may be sufficient space available. This is because there may be considerable space available in this area such that a separate sheath may be used for insertion. In addition, areas adjacent to the renal arteries, for example, may be used for placing more or less bulky parts of stent device 110. These parts may communicate with other parts by, for example, wires or RF communication apparatus. Sensing elements that are placed on the stent graft may be ‘rolled up’ into the rest of the stent graft when inserted into the sheath, for example, in between the various stent sections. In some embodiments, rigid elements which are to be placed in stent graft 105 may be only partially rigid, for example, some elements may be segmented into a series of parts, for example, arranged in a rigid-compliant-rigid-compliant-rigid-etc. order. In some cases measurements may be taken at selected positions around the circumference of the vessel. According to an embodiment of the present invention, stent device components may be placed in a ‘stocking’-like structure that may be attached to the stent graft. Such a stocking-like structure may be made of expandable material that may comply with a vessel's expansion.

The sheaths that may be used for delivery of stent grafts may come in many different diameters, and may have room for varying amounts and/or sizes of components. Furthermore, room may be provided in front of or behind the stent graft. A separate sheath may alternatively or additionally be used for insertion into an aneurysm. According to some embodiments of the present invention, a ring around the graft may enable accumulation of leaks on a drop-by-drop basis. Such a mechanism may prevent the leaks from reaching the aneurism, and may enable a pressure measurement based on the accumulated amount of blood. For example, an elastomer stocking, which may be molded into a required shape, may be used. A elastomer flange, for example, may press more forcefully against the vessel wall than the surrounding parts of the elastomer stocking, thereby stopping blood drops and/or collecting blood drops etc. for measurement. During expansion of the stent, the actual (mechanical) pressure from the vessel wall may increase. This may displace the blood and create a pressure barrier that may stop the blood from being transported. The elastomer flange may, for example, stop bleeding in the aneurism, by setting up a differential pressure that can be detected mechanically.

To minimize the surface areas of apparatus 110 or components thereof, for example, when the device is used for applications requiring high accuracy, surface micro-machining may be used. For example, rapid deep reactive ion etching (DRIE), which is currently available with etching speeds of up to 20 micrometers/min., may be used. Such etching may enable even large volume manufacturing of devices that require partial or complete through-wafer cavities or holes. An example of equipment to implement such a method is produced by Alcatel (Paris, France), for example, in the SINTEF fabrication facility (Oslo, Norway).

Wafer thinning may, for example, incorporate triple-stack glass/silicon/glass wafer stack technology from SensoNor. Such a stacking technology, or other suitable stacking technology may be used in device 110, to enable low cost packaging and provide long-term stability. The triple-stack, however, may make the device thicker. In some embodiments, therefore, the glass wafers may be omitted, thereby providing results that are in line with results from more common surface delivery processes. When the top glass in the SensoNor process is removed, for example, metal lines may be exposed, just as in a surface process. These lines, which may be aluminum lines, may need to be covered by some passivation layer, like nitride. Wafers may be thinned after processing, by for example, Cellular Multiprocessing (CMP) or other suitable operations. Thinning is typically limited by the robustness of the wafers. Thinning may reduce the area advantage of surface machining technologies. Wafers can currently be thinned from standard thickness of around 400 micrometers to about 100 micrometers, although alternative wafer dimensions may be used.

In the case of a pressure sensing membrane, such a membrane, for example, may not be larger than about 100×100 micrometers. The determining factor for the area of the sensing membrane may be the number and/or size of bonding pads being used. The number of bonding pads needed may typically determine the length of the relevant component. For example, a length of 1 mm may provide a sufficient number of pads for most purposes. Pad size may be further reduced by using flip-chip technologies.

The sizes of stent device 110 and the various components may also be effected by ASIC 140 size and by components, e.g., inductors etc., for remote powering and readout. Alternative technologies for flexible mounting of devices may be integrated in some embodiments. It is to be understood that mechanism and/or circuits of device 110 may be integrated into stent graft 105. Alternatively, various components of device 110 may be provided separately from stent graft 105. Any other components or combinations of components may be used.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1. A stent device to monitor parameters related to an endoluminal stent graft, comprising:
at least one compliance sensor to measure compliance between a stent graft and a lumen.

2. The device of claim 1, comprising at least one pressure sensor to measure endoluminal pressure.

3. The device of claim 2, wherein said at least one pressure sensor is adapted to measure pressure at an aneurism of said lumen.

4. The device of claim 2, wherein said at least one pressure sensor is adapted to measure endoluminal pressure using functions selected from the group consisting of blood freshness detection, electrochemical detection, blood color intensity, and blood color shade.

5. The device of claim 1, comprising a transmitter to transmit signals from said stent device to an external unit.

6. The device of claim 1, comprising a controller to control monitoring of parameters related to the endoluminal stent graft.

7. The device of claim 1, comprising an antenna to receive signals from an external unit.

8. The device of claim 7, wherein said antenna is configured to receive energy from an external power source.

9. The device of claim 1, further comprising at least one expansion mechanism which, when activated, is adapted to enable the stent graft to conform to the morphology of said lumen.

10. The device of claim 2, wherein said pressure sensor is selected from the group consisting of a J-shaped tube, and a strain measurement mechanism.

11. The device of claim 1, wherein said compliance sensor comprises a sensor connected to a pressure-sensing chamber located between two sections of a squeezable tube.

12. The device of claim 1, wherein said compliance sensor is adapted to measure parameters selected from the group consisting of compliance between the endoluminal stent and a lumen wall, strain on the endoluminal stent, expansion of an aneurism, pressure on the endoluminal stent, pressure inside the lumen, and pressure outside the lumen.

13. A stent device comprising:
a stent graft; and
at least one anchoring mechanism to attach said stent graft to an aneurism section of a vessel wall.

14. The device of claim 13, wherein said at least one anchoring mechanism comprises at least one anchoring element selected from the group consisting of hooks, wire mesh, glues and polymers to be embedded in a vessel wall.

15. An expandable stent device comprising:
a stent graft; and
at least one expansion mechanism associated with said stent graft and adapted to be activated in response to an external trigger, thereby to adjust the shape of the stent device to correct adverse stent-related conditions at a desired time after initial implantation of the stent device.

16. The device of claim 15, wherein said stent activator activates said at least one expansion mechanism using energy selected from the group consisting of heat, acoustic energy, energy from a chemical reaction, and data signals.

17. The device of claim 15, wherein said at least one expansion mechanism comprises at least one mechanism selected from the group consisting of a neck expansion mechanism, a body expansion mechanism, and an anchoring mechanism.

18. The device of claim 15, wherein said expansion mechanism comprises an expandable element configured to conform to the morphology of a host vessel.

19. The device of claim 15, wherein said expansion mechanism comprises an expandable element configured to urge said stent device against a wall of a host lumen, thereby to improve compliance between said stent device and said host lumen.

20. The device of claim 15, comprising an antenna, said antenna adapted to receive electromagnetic energy from outside a patient to power the stent device.

21. An endoluminal stent system comprising:
a stent device comprising at least one compliance sensor to measure compliance between a stent graft and a lumen; and
an external unit to receive from said stent device signals responsive to said compliance.

22. The system of claim 21, wherein said external unit is able to process the signals received from said stent device.

23. The system of claim 21, comprising an external stent activator to activate at least one expansion mechanism of said stent device.

24. The system of claim 23, wherein said external stent activator activates at least said expansion mechanism by transmitting energy to said stent device.

25. The system of claim 21, comprising a workstation to activate at least one expansion mechanism of said stent device.

26. The system of claim 21, wherein said external is able to supply energy to said stent device.

27. A method to monitor an aneurism, comprising measuring compliance between a stent graft and a lumen.

28. The method of claim 27, comprising transmitting a signal including data relating to said compliance.

29. The method of claim 27, comprising measuring endoluminal pressure.

30. The method of claim 27, comprising processing monitored data relating to said compliance.

31. The method of claim 27, comprising receiving energy from an external power source.

32. A method to externally operate an endoluminal stent after initial implantation of the stent, comprising:
activating at a desired future time after installation of said stent an expansion mechanism associated with said stent by externally triggering an activation trigger associated with said expansion mechanism.

33. The method of claim 32, wherein said expansion mechanism is selected from the group consisting of a neck expansion mechanism, a body expansion mechanism, and an anchoring mechanism.

34. The method of claim 32, comprising anchoring said stent device to the specific morphology of a vessel wall.

35. The method of claim 32, wherein activating said expansion mechanism facilitates functions selected from the group consisting of preventing stent migration, slowing aneurism growth, and treating endoleaks.
